(19) United States
(12) Patent Application Publication
(10) Pub. No.: US 2009/0149963 A1
(43) Pub. Date: Jun. 11, 2009

(54) PROSTHESIS ASSEMBLY INCLUDING ANGLE AND POSITION ADAPTORS

(76) Inventor: Ronald Sekel, New South Wales (AU)

Correspondence Address: STITES & HARBISON PLLC, 1199 NORTH FAIRFAX STREET, SUITE 900, ALEXANDRIA, VA, 22314, US

(21) Appl. No.: 12/093,687

(22) PCT Filed: Nov. 14, 2006
(86) PCT No.: PCT/AU2006/001688

§ 371 Date: Sep. 4, 2008

(30) Foreign Application Priority Data
Nov. 14, 2005 (AU) 2005906273

(51) Int. Cl. A61F 2/38

(52) U.S. Cl. 623/20.15; 623/20.34; 623/20.32

(57) ABSTRACT

A modular prosthesis assembly for insertion in bone, the assembly comprising: a tray element having a first surface including an extension member depending therefrom and an opposite second surface; a first adaptor having first and second ends, the first end having a profile part which receives and retains the extension member; a second adaptor comprising, a first end and a second end; the first end engagable with the second end of the first adaptor; a stem having first and second end, the first end including a formation which allows engagement of the stem with the second end of the second adaptor. The second end of said stem has a profile suitable for insertion in bone.

### BACKGROUND

The present invention relates to improvements in surgical prostheses and more particularly relates to a prosthesis assembly including a tibial tray, mutually interacting angled adaptor elements, a position adaptor and stem.

More Particularly the present invention relates to an assembly which allows fine adjustment of a tibial plate through multiple axes thereby enabling adjustments of a required attitude of the tibial plate. The invention allows fine adjustability of at least one component of the prosthesis assembly through multiple degrees of freedom including; rotation about a Z axis, longitudinal adjustment along an X and Y axes, and vertical adjustment along the Z axis. Although the assembly will primarily be described with reference to its application in adjustment of knee prostheses and particularly tibial components, it will be appreciated by persons skilled in the art that the combination angle adaptor and offset position adaptor is usable in other anatomical bone sites.

### PRIOR ART

Knee arthroplasty is a well-known surgical procedure by which a diseased and/or damaged natural knee joint is replaced by a prosthetic knee joint. Typical knee prostheses include a tibial component, a femoral component, and a patellar component. Modern total knee replacement involves the resurfacing of the femoral condyles with a metallic component, roughly approximating the shape of the anatomical femoral condyles, and resurfacing the tibial plateau with usually, but not exclusively, a polyethylene component having a metallic tibial base plate. Ideally the femoral component should be congruent with the top of the tibial component in order to minimise wear of a surface liner which is usually polyethylene. During normal movements of the knee, rotation of the femur upon the tibia occurs, and roll back of the femoral condyles upon the tibia occurs, particularly when the knee is flexed in normal rear flexure.

The femoral component generally includes a pair of spaced apart condylar portions, the superior surfaces of which articulate with a portion of the polyethylene tibial component. A femoral stem assembly, used to provide lateral stability to the replaced knee joint, engages and seats within the medullary cavity of a distal portion of a femur, and is typically fixed to the femoral component by specialized fixation, such as a keel or a collar and bolt. Some prosthetic knee joints include a taper which may be a Morse type taper, that extends from the back surface of the femoral component to mate with a femoral sleeve that is securable to the femoral stem assembly.

Although the femoral stem, is usually angled with respect to the inferior surface of the femoral component and either off-set anteriorly/posteriorly or at a central location, it is sometimes desirable to orient the femoral stem perpendicularly with respect to the back surface. The femoral stem may need to be offset fore or aft with respect to the front of the femoral component. Similarly, the femoral stem may need to be angled varying degrees to the left or right with respect to the front plane of the femoral component. A Morse type taper post, is integrally cast as part of the femoral component.

The tibial plate component also typically requires similar orientation adjustments to its angular disposition left or right, anteriorly or posteriorly. Despite the existence of knee joint prostheses having modular components, there remains a need for a modular knee joint prosthesis assembly that has greater versatility of adjustment to accommodate differing patient anatomy and a misaligned components to a greater extent that that already available.

An example of a known knee prosthesis arrangement is disclosed in U.S. Pat. No. 5,593,449 to Robertson Jr. That patent discloses a dual taper stem extension for knee prosthesis for surgical implantation to a patient's leg bone at the knee joint area. The prosthesis includes a prothesis body portion that extends transversely relative to the patient's intramedullary canal for carrying a bearing surface that articulates with the patient's adjacent leg bone or with another prosthesis component. A conical connector extends from the prosthesis portion and along an axis that generally tracks the patient's intramedullary canal. A stem member includes first and second end portions and has a central longitudinal stem axis. The stem member includes a socket at each end portion for forming connections to the conical connector at the respective end portions as selected by the surgeon. One of the sockets has a central longitudinal axis that generally coincides with the central longitudinal axis of the stem. The other socket has a central longitudinal axis that forms an acute angle with the axis of the stem. The arrangement disclosed in this patent allows the surgeon to select from a choice of two taper angles the valgus angle for a stem extension that will best fit the patients intramedullary canal but once the angle is selected the coupling allows only two degrees of freedom i.e. axial and rotational movement.

Another known knee prosthesis is disclosed in U.S. Pat. No. 5,782,921 to Colleran which teaches a modular knee prosthesis including a Morse taper post that is matable with a first portion of a femoral sleeve. A second portion of the femoral sleeve is joined with a femoral stem that is introducible within the medullary canal of a distal portion of a femur. The modular knee prosthesis includes a femoral component, a bolt, and a Morse taper post. The femoral component has a superior surface, an inferior surface, and an aperture. The bolt includes a head portion engagable with the superior surface of the femoral component to inhibit movement of the bolt through the femoral component, and an elongate shaft portion that extends from the head portion of the bolt. The elongate shaft portion has a length sufficient to protrude through the aperture beyond the inferior surface of the femoral component. The Morse taper post is engagable with the elongate shaft portion of the bolt to retain the Morse taper post in a fixed position with respect to the femoral component and the distal end of the Morse taper post is introducible within a femoral sleeve. This assembly has some degrees of freedom of adjustment but the extent of adjustment is limited to the adjustments in known knee assemblies.

Typically, a knee prosthesis will comprise a femoral component for securing to the femur, an opening defined by the femoral component, a tibial component for securement to the tibia, an opening through the tibial component, a bearing component between the femoral and tibial components, the femoral component and the bearing component having respective curved articulatory cooperating bearing surfaces.

Examples of resurfacing types of total knee prosthetic devices are also disclosed in the following US patents incorporated by reference herein. U.S. Pat. No. 3,774,244 to Walker; U.S. Pat. No. 3,728,742 to Averill et al. U.S. Pat. No. 4,081,866 and U.S. Pat. No. 4,207,627 to Cloutier.

Various knee prosthesis components include elongate stems that are to be mounted within the intramedullary canal of a bone while the other end is attached to another prosthesis component that is mounted in opposing bone. The large variation in the human anatomy of different patients creates the need for a variety of implant sizes and configurations. In some cases, it is necessary for the longitudinal axis of the stem to be laterally offset from the longitudinal axis of other prosthesis components. The tibial component usually comprises a tibial stem which is attachable to a tibial tray. The tibial stem is designed to be installed within the intramedullary canal of the tibia while the tibial tray mounts upon a prepared surface on the head of the tibia. A tibial bearing member, which articulates with the femoral component, is typically mounted upon the tibial tray.

Since the tibia exhibits great variation among patients, some knee arthroplasty patients may require that the tibial stem prosthesis component be implanted in an orientation such that the longitudinal axis of the tibial stem is colinear with the longitudinal axis of the tibial tray prosthesis component. In other patients these axes must be offset with respect to one another to ensure proper implantation of the tibial stem and simulation of the patients natural anatomy.

Modular tibial prosthesis systems and assemblies have been developed to accommodate the variability in patient anatomies. Modular systems include a number of interchangeable parts, each having different sizes or other physical characteristics. Modular systems allow surgeons to use one or more standard parts with interchangeable components having different characteristics.

For example, U.S. Pat. No. 5,290,313 discloses a modular tibial prothesis in which a tibial stem is mounted so as to be laterally offset with respect to the longitudinal axis of a tibial tray. A coupling allows specially designed tibial stems to be mounted to the tibial tray to achieve a desired offset orientation. One disadvantage of this design is that the tibial stems themselves are offset, and a different stem must be used to achieve a desired offset orientation. As a result, a number of different, non-standard tibial stems are needed to achieve the desired offset orientation required for a given patient. Such a system can increase the cost of prostheses because several non-standard parts are necessary to cope with all possible anatomical requirements of patients.

Modular joint prosthesis components are needed which optimize the fit within the patient while, at the same time, allowing greater flexibility to the surgeon for accurate and proper setting of the bearing plate. It is also desirable to be able to achieve optimal fit of prosthesis components while still reducing the inventory of joint prosthesis parts that are needed to meet patient needs.

U.S. Pat. No. 5,782,920 discloses an offset coupling for a joint prosthesis which allows an inferior component of a prosthesis system to be offset from a superior component of the system. In one embodiment the joint prothesis component system comprises a tibial tray having an offset tibial stem. An adapter element connects between the tibial tray and tibial stem to provide a desired degree of offset and the orientation of the offset. The adapter element is constructed such that a longitudinal axis extending through a first end thereof is offset from a longitudinal axis extending through a second end thereof.

The anatomy of the human tibia is variable. The tibia comprises an outer layer of hard cortical bone and an inner filling of relatively soft cancellous bone. The strength of the tibia is primarily derived from the cortical bone and should be preserved to support implants. Normally, the intermedullary canal is not in the actual center of the proximal tibia. As a result, when the bore is formed using the intramedullary canal, the stem which is placed in the bore may not be centered within the proximal tibia. Therefore, because the baseplate is mounted on the stem, the baseplate may not be ideally positioned with respect to the resected tibial surface. This can result in an overhang of the baseplate relative to the resected end of the tibia and a resulting irritation of soft surrounding tissue. The baseplate must therefore be kept within the confines of the tibia. One solution to this problem has been to provide an offset, either medially or laterally, in order to properly orient the baseplate on the proximal tibia relative to the stem.

Where a press fit tibial stem is used, a surgeon reams out the intramedullary canal and removes cancellous bone. Subsequently, the surgeon trials with various size straight tibial stems having a baseplate attached, to find a stable press-fit with the cortical bone. If the bore is offset to compensate for the off-center canal within the tibia, the implant may be supported on some surface areas by the hard cortical bone and on other surface areas by the relatively softer cancellous bone. This is undesirable.

One attempt to overcome the above-mentioned problems with tibial stem implants is addressed in U.S. Pat. No. 5,133,760, which provides a universal modular prosthetic stem extension which may be installed on a prosthesis in a multiplicity of different orientations to compensate for a multiplicity of patient conditions. The stem includes a coupling mechanism allowing the stem to be rotated to any one of a multiplicity of rotative positions with respect to a prosthetic base so that the stem may be fixed in position relative to the base.

Another device is disclosed in U.S. Pat. No. 5,271,737 which comprises a combination baseplate fixed to an offset, straight tibial stem. The base includes an inferior surface for abutting a resected surface of the patient's tibia. The longitudinal center axis of the straight tibial stem extends from the inferior surface of the base and is offset from a center of the base. The offset places the stem in position to extend into the canal of the tibia so that it does not interfere with the cortical bone. As a result of the fixed arrangement, one baseplate and stem is required for a medial offset and another is required for a lateral offset.

A further assembly is disclosed in U.S. Pat. No. 5,290,313, which comprises a modular prosthesis system including a modular stem which has an attachment section for attachment to the base, a main body section for implanting into the canal in the tibia, and an angled transition section. The attachment section and main body section each include a respective longitudinal axis. These axes are parallel to each other and spaced apart to provide an offset therebetween. The offset is substantial, such that the axis of the attachment section intersects the transition section.

In another example of the prior art, U.S. Pat. No. 6,146,424 discloses another offset, press-fit tibial stem system including a baseplate, a main body portion, a baseplate connection portion attached to the baseplate and being offset from, and connected to, the main body portion by a transition portion. The main body portion has a first centroidal axis passing therethrough and the baseplate connection portion has a second centroidal axis passing therethrough. The offset is such that the first centroidal axis and the second centroidal axis are slightly spaced apart and substantially parallel so as to each pass through the main body portion.

Although the issue of and need for greater versatility of adjustment of prostheses has been addressed in a number of prior art arrangements such as those described above, there is still a need to increase the fine adjustability of artificial joints relative to orthogonal X Y and Z axes and rotationally through multiple three dimensional degrees of freedom to more easily compensate for unwanted misalignments and to ensure that an implanted prosthesis conforms as close as possible to the natural anatomy of the patient that the joint is replacing.

Typically integral with the Tibial plate is a stem adapted for insertion in a medullary cavity of tibial bone. The stem is friction fitted and may be cemented into a suitably reamed medullary cavity. However if the reamed cavity is inaccurately formed, the tibial plate (or corresponding femoral component) may sit at an angle relative to a bone section cut by the surgeon as a reference prior to insertion of the tibial component. Once the known tibial component is inserted, the currently preferred way a correcting alignment adjustment may be made is to remove the tibial component and try to reset it. This is an undesirable solution to misalignment as a refit will possibly result in a potentially weaker bone/component bond. The setting of the stem must therefore be highly accurate to avoid the problems of misalignments. In practice, most insertions require some angular or rotational adjustments

The present applicant has earlier addressed the aforesaid problems of the prior art devices in PCT application No. PCT/AU03/00122 entitled: Modular Prosthesis Assembly With Adjustable Taper which is incorporated by reference herein.

The arrangements disclosed in that application increase options for angular adjustment.

### INVENTION

There is a further need to provide a convenient and effective means for fine adjustments of prostheses where an initial fit is not in conformity with alignment parameters. For example, in the case of a tibial component of a knee prosthesis, the tibial plate may not align with a patient reference plane due for instance to the setting in the medullary cavity. The misalignment may be in one or more planes or in one or more axes. Accurate fixation of the tibial component to ensure proper alignment is a difficult surgical objective particularly due to the difficulty in accurately preparing the medullary cavity in the tibia. It is an advantage for a surgeon to be able to make fine axial, rotational, lateral and anterior/posterior adjustments through multiple planes and axes as this would allow correction of any misalignments or non conformity with insertion parameters.

The present invention provides an assembly including a tibial plate, an angle adaptor and position adaptor which fits a distal stem and allows a surgeon to make fine adjustments to a tibial component to best simulate normal anatomy. More particularly the present invention provides an assembly which combines a position adaptor presenting a proximal end to an angle adaptor and which may be rotated through 360 degrees resulting in an offset from a longitudinal axis passing through a distal end of the adaptor. Preferably the adaptor allows rotational position adjustments which provide corrections preferably at least up to 10 mm, laterally anteriorly and posteriorly by angular rotation relative to a stem.

The assembly is potentially capable of use with a variety of bone and skeletal joint prosthesis. For instance the assembly and associated angle adaptor and position adaptor may be applied in effecting fine adjustments to dental fixations, tibial and femoral implants (distal or proximal), ankles, fingers and a variety of other joints and bone sites.

It is therefore an object of the invention to provide a modular prosthesis assembly including a position adaptor which allows increased versatility of adjustment to accommodate predetermined insertion parameters, simulate patient anatomy, joint attitude and conditions while maintaining a relatively low assembly component count.

It is another object of the invention to provide a modular prosthesis assembly including components that are physiologically and geometrically compatible with different anatomical conditions and which allow selective offset adjustments by rotation of a position adaptor.

In one broad form the present invention comprises:

A modular prosthesis assembly comprising; an anchorage component insertable in a bone cavity,

a position adaptor for coupling to the anchorage component and having a first end which is off set from a longitudinal axis of the anchorage component and a second end in alignment with said axis and which engages the component so as to allow up to 360 degrees of relative rotation therebetween;

an angle adaptor having a longitudinal axis and first and second ends, the second end engaging the first end of the position adaptor and allowing up to 360 degrees of rotation relative to the position adaptor prior to fixed engagement between the angle adaptor and the position adaptor, the first end of the angle adaptor including a recess disposed at an angle to its longitudinal axis,

a tray including an extension stem which is received and retained by said angled recess; wherein the tray is rotatable through up to 360 degrees relative to the angle adaptor; wherein engagement between the position adaptor and stem and the angle adaptor and position adaptor and between said extension stem and angle adaptor allow the tray to undergo fine adjustments including rotation in an X plane and Y plane and rotation about a Z axis.

In another broad form the present invention comprises:

a modular tibial prosthesis assembly, comprising:

a tray element having a first surface that is mountable upon bone and a second, opposed surface, the first surface including an extension member depending therefrom;

an angle adapter element having first and second ends wherein the first end comprises an opening which receives and retains therein the extension member;

a position adaptor comprising, a first end and a second end; a first longitudinal axis extending through the first end which is substantially parallel to but offset from a second longitudinal axis extending through the second end, the first end having a recess matable with the second end of the angle adaptor; the second end adapted for mating with an elongate stem mountable within bone, the elongate stem having a first end that is selectively matable with the second end of the position adaptor; wherein the angle adaptor allows angle adjustments and the position adaptor allows rotational positional adjustments effective to secure the position adapter in a desired orientation when mated with the angle adaptor and stem.

Preferably the tray element is rotatable within said angle adaptor and the angle adaptor is rotatable in a first end recess of the position adaptor.

In another broad form the present invention comprises:

a positional adaptor for use with a modular tibial prosthesis assembly, the assembly comprising:

a tray element having a first surface that is mountable upon bone and a second, opposed surface, the first surface including an extension member depending therefrom;

an angle adapter element having first and second ends wherein the first end comprises an opening which receives and retains therein the extension member;

the position adaptor comprising, a first end and a second end; a first longitudinal axis extending through the first end which is substantially parallel to but offset from a second longitudinal axis extending through the second end, the first end having a recess matable with the second end of the angle adaptor; the second end adapted for mating with an elongate stem mountable within bone, the elongate stem having a first end that is selectively matable with the second end of the position adaptor; wherein the angle adaptor allows angle adjustments and the position adaptor allows rotational positional adjustments effective to secure the position adapter in a desired orientation when mated with the angle adaptor and stem.

In another broad form the present invention comprises:

a positional adaptor for use with a modular tibial prosthesis assembly, the assembly comprising:

a tray element having a first surface that is mountable upon bone and a second, opposed surface, the first surface including an extension member depending therefrom;

the position adaptor matable with said extension member and comprising, a first end and a second end; a first longitudinal axis extending through the first end which is substantially parallel to but offset from a second longitudinal axis extending through the second end, the first end having a recess matable with the extension member the second end adapted for mating with an elongate stem mountable within bone, the elongate stem having a first end that is selectively matable with the second end of the position adaptor; wherein the position adaptor allows rotational positional adjustments effective to secure the position adapter in a desired orientation when mated with the stem.

Preferably the second end of the positional adaptor mates with the stem in a male female relationship.

Preferably the first end of the angle adaptor has a female recess and the second end is a male profile part. Preferably the tray is a tibial tray and the extension member is a tibial stem. Preferably the first and second longitudinal axes of the position adaptor are offset from each other by approximately 1 to 10 mm.

Preferably, the first and second ends of the position adaptor are generally cylindrical, the first end having a tapered recess and the second end having a male profile part. The tapered recess is according to one embodiment, of corresponding shape to the extension member of the tibial plate.

In another broad form the present invention comprises:

A modular prosthesis assembly for insertion in bone, the assembly comprising:

a tray element having a first surface including an extension member depending therefrom and an opposite second surface;

a first adaptor having first and second ends, the first end having a profile part which receives and retains the extension member;

a second adaptor comprising, a first end and a second end; the first end engagable with the second end of the first adaptor,

a stem having first and second end, the first end including a formation which allows engagement of said stem with the second end of said second adaptor,

the second end of said stem having a profile suitable for insertion in bone.

Although the invention will be primarily described with reference to its application to knee prostheses and particularly a tibial implant, it will be recognised by persons skilled in the art that the positional adaptor and associated taper arrangements described herein which allow multiple degrees of freedom for fine adjustments to the attitude of a component, may be applied in other prostheses such as may be used to repair fingers, thumbs, shoulders and ankles. The assembly may also be used in dental applications where a component is used to anchor an artificial tooth to a jaw bone. It will be appreciated by persons skilled in the art that tapers other than a Morse type taper may be used on the assembly and adaptors according to the invention. Also alternative mating geometry is contemplated including polygonal including hexagonal mating profile parts.

According to another embodiment, the prosthesis assembly comprises an anchoring member insertable in a bone cavity, a tibial component which is capable of mating with the anchoring member; an adaptor capable of co operating with said anchoring member and the tibial component to allow fine adjustment of the tibial component.

Preferably, the positioning adaptor includes a body having an external tapered region at a second end and a tapered inner recess at a first end, wherein the external tapered region releasably engages a stem and the inner tapered recess receives therein an extension member of a tibial plate. The positing adaptor allows lateral and anterior and posterior adjustment through about 1-6 mm although it will be appreciated by those skilled in the art that the extent of offset correcting adjustment could exceed 6 mm.

The positional adaptor is preferably adjustable rotationally and axially with the rotational adjustment providing an orthogonal displacement from a longitudinal axis of the stem.

According to one embodiment, the inner recess of the position adaptor is a taper is coaxial with an external taper of an angle adaptor.

The inner taper may be offset relative to but parallel to a longitudinal axis of said adaptor or the inner taper may be offset from and at an angle relative to a longitudinal axis of the adaptor.

In another broad form, the present invention comprises;

a modular prosthesis assembly comprising; an anchorage component insertable in bone and a coupling component which cooperates with said anchorage component to assume a first predetermined orientation relative to said anchorage component; the assembly further comprising an adaptor insertable between said anchorage component and said coupling component to allow a secondary adjustment of said coupling component relative to said first predetermined orientation of said coupling component.

Preferably, the adaptor is coupled to the tibial extension via male/female engagement.

Preferably, the adaptor is coupled to the stem male/female engagement.

In another broad form according to a method aspect the present invention comprises:

a method of insertion of a modular tibial prosthesis assembly, comprising the steps of

a) taking a tray element having a first surface that is mountable upon bone and a seconds opposed surface, the first surface including an extension member depending therefrom;

b) taking an angle adapter element having first and second ends wherein the first end comprises an opening which receives and retains therein the extension member;

c) taking a position adaptor comprising, a first end and a second end; a first longitudinal axis extending through the first end which is substantially parallel to but offset from a second longitudinal axis extending through the second end,

d) taking a stem capable of insertion in bone;

d) inserting the stem in a medullary cavity of a tibia;

e) inserting the positional adaptor via its second end into the stem, the elongate stem having a first end that is selectively matable with the second end of the position adaptor,

f) placing the angle adaptor via its second end in a recess is the first end of the position adaptor

g) inserting the extension member of the tibial plate into a recess in said angle adaptor via its first end

h) adjusting the tibial plate by selective adjustment of the angle adaptor and the position adaptor to orientate the tibial plate;

i) adjusting the position adaptor within an arc of 0-360 degrees.

wherein the angle adaptor allows angle adjustments and the position adaptor allows rotational positional adjustments effective to secure the tibial plate in a desired orientation when mated with the assembly.

Preferably the method comprises the following additional steps:

a) checking the orientation of the tibial plate relative to a predetermined anatomical reference once the angle adaptor and position adaptor have been assembled;

b) in the event that the first orientation of the tibial plate is incorrect relative to said anatomical reference, resetting said plate and either the angle and/or position adaptor so the tibial plate assumes a secondary disposition which is a preferred orientation relative to a predetermined anatomical reference.

### BRIEF DESCRIPTION OF THE DRAWINGS

The present invention will be now described according to a preferred but non limiting embodiment and with reference to the accompanying illustrations wherein

FIG. 1 shows an exploded view of a tibial prosthesis assembly according to one embodiment

FIG. 2 shows a cross sectional view of the assembly of FIG. 1.

FIG. 3 shows an exploded view of a tibial prosthesis assembly according to an alternative embodiment

FIG. 4 shows a cross sectional view of the assembly of FIG. 3.

FIG. 5 shows an elevation view of an angle adaptor according to one embodiment isolated from the assembly.

FIG. 6 shows an elevation view of the angle adaptor of FIG. 5 rotated ninety degrees.

FIG. 7 shows a cross sectional view of the angle adaptor of FIG. 6 taken along line A-A.

FIG. 8 shows an elevation view of a position adaptor according to one embodiment isolated from the assembly.

FIG. 9 shows an elevation view of the position adaptor of FIG. 8 rotated ninety degrees.

FIG. 10 shows a cross sectional view of the angle adaptor of FIG. 9 taken along line A-A.

FIG. 11 shows a fully assembled prosthesis assembly according to one embodiment inserted in a tibia.

FIG. 12 shows the fully assembled prosthesis assembly of FIG. 11 according to one embodiment inserted in a tibia.

The invention will be primarily described with reference to its application in knee prostheses. It will be appreciated however, that the assembly described herein including the use of an angle adaptor for angular adjustment and a position adaptor for lateral offset for re adjustment of a component may be applied in a variety of skeletal sites including but not limited to shoulder, ankle, finger, thumb joints. Also the assembly may be employed in dental applications. In known total knee prostheses the articular surface of the distal femur and proximal tibia are usually but not exclusively replaced with respective metal and plastic condylar-type articular bearing components. The knee prostheses provides adequate rotational and translational freedom and require minimal bone resection to accommodate the components within the boundaries of the available joint space. The stem of the tibial assembly components is affixed to respective, surgically prepared adjacent bone structure by cementing or by biological bone ingrowth. The tibial component can be made entirely of ultra high molecular weight polyethylene or can be comprised of a metallic base and stem component distally and an interlocking plastic (UHMWPE) component, proximally. The plastic tibial tray plateau bearing surfaces are of concave multi-radius geometry.

The objective in knee replacements is to simulate with a dynamic implant, natural knee function as closely as possible and any improvement which allows a surgeon greater flexibility in achieving this objective is desirable.

The present invention described herein with reference to alternative embodiments, provides a prosthesis assembly including an angle adaptor and position adaptor which enables a surgeon to make finer adjustments to the disposition or attitude of a tibial component than has previously been possible to enable the orientation of the tibial plate to more accurate simulate anatomical joint geometry.

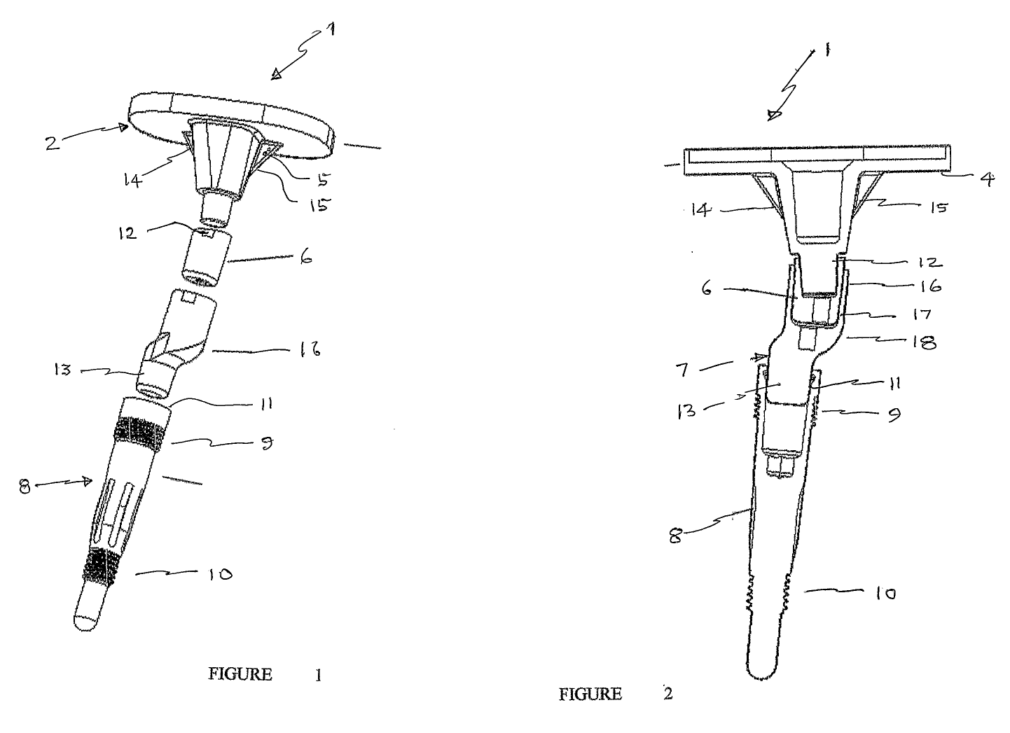

FIG. 1 shows a perspective exploded view of a typical tibial component assembly 1 and FIG. 2 shows a cross sectional view of the assembly of FIG. 1. Assembly 1 comprises a tibial tray or plate 2 and a tibial extension member 3. An underside surface 4 of plate 2 may be adapted with a porous coating 5 with or without the use of a bone growth promoter Hydroxyapatite. Alternatively, the underside surface 4 of plate 2 may be roughened by grit blasting.

Extension member 3 of plate 2 engages angle adaptor 6 via recess 12 (see FIG. 2) such that a longitudinal axis of extension member 3 is either coaxial with or depending upon the orientation of the recess 12, intersects a longitudinal axis of angle adaptor 6. Preferably recess 12 is angled within the region 0-6 degrees.

Tibial tray/plate 2 when set in position may be out of alignment with an anatomical reference such as a tibial plateau. In that case, where the surgeon anticipates the possibility of an out of alignment of plate 2, angle adaptor 6 and a position adaptor 7 may be used to make fine adjustments to plate 2 both angularly, rotationally axially, laterally, posteriorly and anteriorly.

Assembly 1 further comprise a stem 8 which is a known distal shaft used in fixation to bone. In the embodiment shown, stem 8 having double threaded regions 9 and 10 provide anchorage in the tibia (not shown). Stem 8 also includes recess 11 (see FIG. 2) which receives and retains therein end 13 of position adaptor 7. End 13 is preferably a female profile part but in an alternative embodiment this may be a reverse gender connection; i.e. female/male.

When assembled, tibial plate 2 engages via extension member 3 recess 12 in angle 6. Locating wings 14 and 15 allow anchorage of tibial plate 2 into a bone to prevent unwanted rotational movement. Recess 12 is preferably tapered but may be other geometric shapes to accommodate alternative cross sectional shapes of extension 3. When recess 12 receives and retains therein tibial extension 3, this will dictate the initial orientation in situ of tibial plate 2 relative to an anatomical reference.

Ideally when in situ, tibial plate 2 will be parallel with a bone plateau prepared by the surgeon prior to fixation of assembly 1. However, this is not always the case sometimes due to poor technique and the surgeon has not previously until the present invention been able to make all possible fine adjustments for the orientation of the tibial plate once it has been inserted. Accurate insertion of the assembly may be inhibited by a patients bone condition or the manner of reaming of the medullary cavity prior to insertion. Errors in reaming may be translated into an error in the disposition of tibial plate 2 which must somehow be corrected to ensure a result which causes the prosthesis to simulate the anatomy of the replaced joint.

In some cases the orientation of tibial plate 2 will be outside an optimum disposition for ultimate simulation by the artificial joint of natural joint geometry and function. The present invention provides an assembly of components including a coacting angle adaptor and a positional adaptor allows a surgeon to make additional fine adjustments in conjunction with those already available in the art to improve the orientation of the tibial plate so it is set in a disposition required relative to a predetermined anatomical or other reference. The surgeon may choose position adaptor 7 having first end 16 and second end 13. First end 16 includes a recess 17 (see FIG. 2) which receives and retains therein known angle adaptor 6. Second (distal) end 13 is preferably a male profile part which engages recess 11 of stem 8. Position adaptor 7 is rotatable in recess 11 (prior to final engagement) and will allow due to an offset region 18 a positional offset of recess 17 and therefore adaptor 6 anywhere within 0-360 degrees about a longitudinal axis through end 13.

Angle adaptor 6 allows the surgeon to conduct a first orientation of plate 2 and subsequent reorientations until the plate is set in position. In addition the surgeon may rotate position adaptor 7 about 360 degrees until the surgeon is satisfied with the final location of the tibial plate. This might require a number of fine adjustments and re adjustments until the optimal position for tibial plate is eventually achieved.

The position adaptor 7 therefore adds additional capacity for adjustment and particularly rotational adjustment during insertion of the tibial assembly 1. This is used in conjunction with the angle adaptor 6.

Adaptor 7 may be used to adjust the length of an implant, the direction of gradient of tibial plate 2 and the rotation about an axis through stem 8.

In another embodiment, alternative anchorage members are used to extend the depth of penetration inside a medullary cavity. In the case of a revision where bone has degraded an allograft may be required. This will normally necessitate a deeper anchorage in the medullary cavity. For this purpose tibial stem 8 may be increased in length.

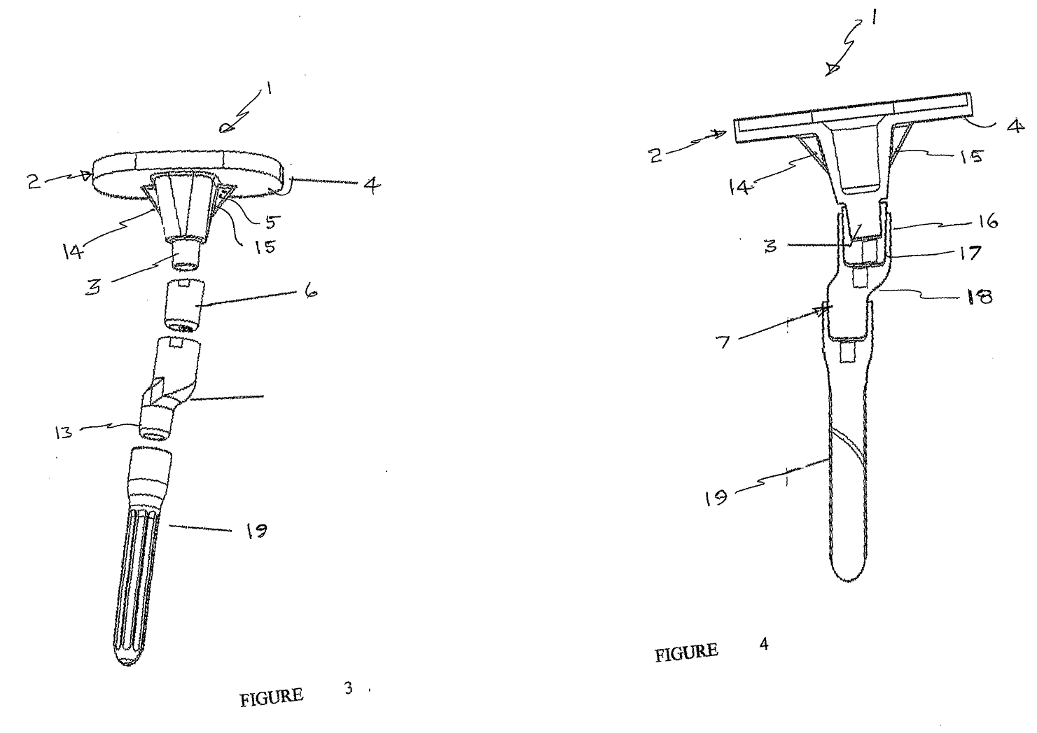

FIG. 3 shows an exploded view of a tibial prosthesis assembly according to an alternative embodiment and FIG. 4 shows a cross sectional view of the assembly of FIG. 3.

The embodiment of FIGS. 3 and 4 is substantially the same as the embodiment of FIGS. 1 and 2 and bears numbers which correspond to like parts. In the embodiment of FIGS. 2 and 3 an alternative fluted stem 19 is adapted to end 13 of positional adaptor 7. Stem 19 has a female recess 20 which mates with end 13 and whose longitudinal axis is coaxial generally in alignment with position adaptor 13. Beyond the use of an alternative stem 19 the assembly of FIGS. 3 and 4 functions as previously described with reference to FIGS. 1 and 2.

It may be seen from FIGS. 2 and 4 that as assembly 1 is fully assembled, that a surgeon has at his disposal a wide scope for fine attitude adjustment of the plate including rotation about X and Y axes, rotation about a Z axis, radial rotation eccentric to a longitudinal axis of the stem and axial adjustments to enable increase in the overall length of the assembly. With an angled recess in adaptor 6 and combined with the rotation of the tibial tray about its own axis and the rotation of the position adaptor about stem 8 or 13 The surgeon has as his disposal wide scope for fine adjustment.

FIG. 5 shows an elevation view of an angle adaptor 30 according to one embodiment isolated from the assembly. FIG. 6 shows an elevation view of the angle adaptor 30 rotated ninety degrees, and FIG. 7 shows a cross sectional view of the angle adaptor 30 taken along line A-A. As best shown from the sectional view, adaptor 30 includes a recess 31 which is disposed at an angle to longitudinal axis 32. The angulation of the recess which will according to a preferred embodiment be between 0-10 degrees. Adaptor 30 also includes an opening 33 which allows fixation of angle adaptor 30 to the positional adaptor 34 (see FIGS. 8-11).

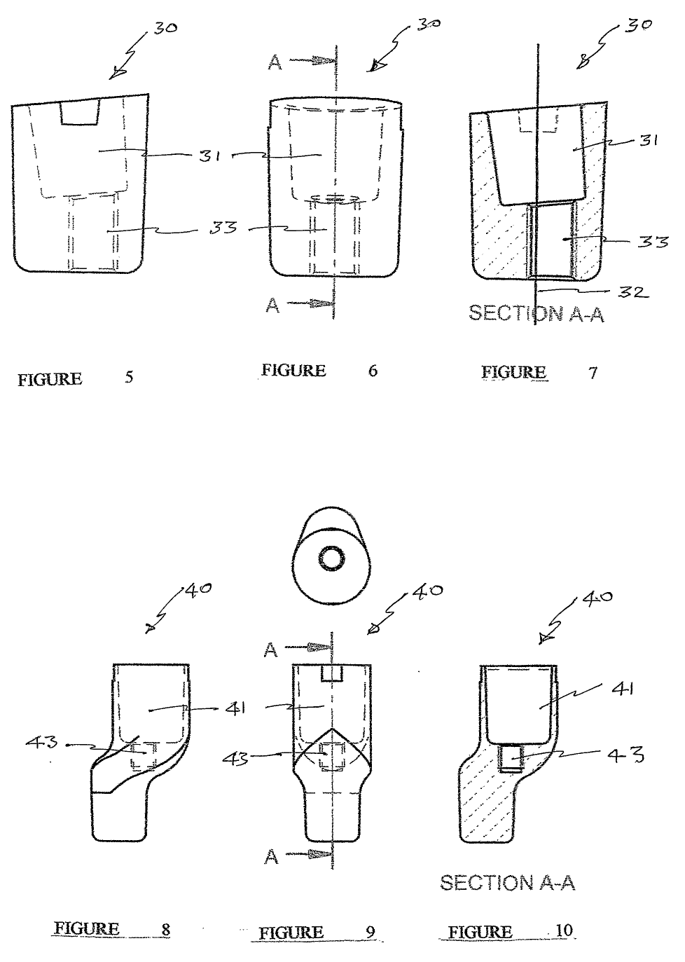

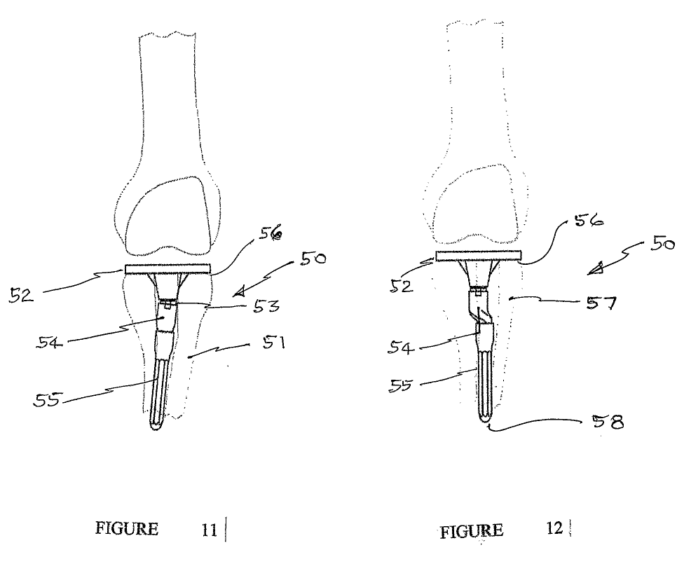

FIG. 8 shows an elevation view of a position adaptor 40 according to one embodiment isolated from the assembly. FIG. 9 shows an elevation view of the position adaptor 40 rotated ninety degrees. FIG. 10 shows a cross sectional view of the angle adaptor 40 taken along line A-A.

As best shown from the sectional view, positional adaptor 40 includes a recess 41 which in use receives adaptor 30 which is able to rotate therewithin. Adaptor 40 also includes an opening 43 which allows a fixation devices such as a screw (not shown) to fix angle adaptor 30 in recess 41 once the rotational position of angel adaptor is set.

FIG. 11 shows a fully assembled prosthesis assembly 50 according to one embodiment inserted in a tibia 51. Assembly 50 includes tray 52, angle adaptor 53 positional adaptor 54 and stem 55. Assembly 50 is shown in a first orientation relative to tibia 51. Tray 52 is shown in a particular setting which is optimal relative to the tibial plateau 56. FIG. 12 shows the fully assembled prosthesis assembly 50 inserted in a tibia 57 but is an alternative orientation relative to medullary cavity 58. In this embodiment, it can be shown that due to the wide scope for adjustment of the prosthesis the plate can adopt a correct disposition on the tibal plateau while the remainder of the assembly beneath can be in different orientations to achieve the correct tray/plate setting. Thus, FIGS. 11 and 12 show two assembly geometries but there are potentially a large number of geometries to achieve correct orientation, with those geometries largely dictated by the alterations and adjustments that the surgeon is required to make to correctly seat the tray on the tibal plateau.

Although the invention according to alternative embodiments, has been described with reference to use of Morse tapers to effect connections, other profile connections may be used such as but not limited to polygonal cross sections including hexagonal

For any joint prosthesis replacement including the knee to function optimally 4 vectors need to be considered in the design to return the joint position in space to as normal as possible a natural position. The four vectors are;
1 medial-lateral
2 anterior-posterior
3 rotational
4 vertical (height).

The assembly described above accommodates all vectors of movement and adds, due to the positional adaptor, a further ability for an offset rotation in conjunction with the adjustments allowed by angle adaptor 6. In an alternative embodiment, the extension stem can be joined on to the tibial component without correction of the adaptor components. The angle adaptor can be used with the extension stem to provide angular correction. The position adaptor can be used with the extension stem to provide radial position correction. The angle and position adaptors can be used with the extension stem to provide both angular and position correction and provide the full scope of fine adjustments available to the surgeon.

It will be recognised by persons skilled in the art that numerous variations and modifications may be made to the invention broadly described herein without departing from the overall spirit and scope of the invention.

1. A modular prosthesis assembly for insertion in bone, the assembly comprising:
a tray element having a first surface including an extension member depending therefrom and an opposite second surface;
a first adaptor having first and second ends, the first end having a profile part which receives and retains the extension member;
a second adaptor comprising, a first end and a second end; the first end engagable with the second end of the first adaptor,
a stem having first and second end, the first end including a formation which allows engagement of said stem with the second end of said second adaptor,
the second end of said stem having a profile suitable for insertion in bone.

2. An assembly according to claim 1, wherein the second end of said second adaptor is offset from alignment with the first end.

3. An assembly according to claim 2 wherein the second adaptor includes a first longitudinal axis extending through its first end and a second longitudinal axis offset from the first longitudinal axis and extending through the second end.

4. An assembly according to claim 3 wherein, the tray is rotatable relative to the first and second adaptors.

5. An assembly according to claim 4 wherein, the first adaptor is rotatable relative to the second adaptor.

6. An assembly according to claim 5 wherein the second adaptor is rotatable relative to the stem.

7. An assembly according to claim 6 wherein the second end of the second adaptor engages the first end of the stem via opposite gender interfitting.

8. An assembly according to claim 7 wherein, when the second adaptor is fitted to said stem, a longitudinal axis of the stem aligns with the second longitudinal axis of the second end of the second adaptor.

9. An assembly according to claim 8 wherein the second adaptor engages the stem via recess in the stem.

10. An assembly according to claim 9 wherein the longitudinal axis of the second end of the second adaptor remains off set from the longitudinal axis of the stem.

11. An assembly according to claim 10 wherein the first adaptor engages via its second end a recess in the first end of the second adaptor.

12. An assembly according to claim 11 wherein the first adaptor is rotatable relative to the second adaptor in said recess of the first end of the second adaptor.

13. An assembly according to claim 12 wherein, the first adaptor has a recess at is first end which receives and retains therein said extension member of said tray.

14. An assembly according to claim 13 wherein, the recess in the first adaptor is disposed at an angle relative to a longitudinal axis of said first adaptor.

15. An assembly according to claim 14 wherein a longitudinal axis of the recess in the first adaptor aligns with a longitudinal axis of said extension member of said tray.

16. An assembly according to claim 15 wherein engagement between said extension member of said tray with said first adaptor and engagement of said first adaptor with said second adaptor allows a plurality of positional adjustments to the orientation of said tray relative to the longitudinal axis of said stem.

17. An assembly according to claim 16 wherein the tray may be rotationally adjusted relative to the stem via the second adaptor and relative to the second adaptor via the first adaptor and relative to the first adaptor via said extension member.

18. An assembly according to claim 17 wherein the tray is a tibial tray for use in an artificial knee.

19. An assembly according to claim 18 wherein the first adaptor allows angle and rotational adjustments to the extension member of the tibial tray.

20. An assembly according to claim 19 wherein the second adaptor allows length and rotational adjustments to the assembly.

21. An assembly according to claim 20 wherein the tray provides a tibial component for an artificial knee.

22. An assembly according to claim 21 wherein the stem is insertable in a medullary cavity of a tibia.

23. An assembly according to claim 22 wherein the longitudinal axis of the second end of the second adaptor is offset 1-10 mm distant from the longitudinal axis of the first end of the second adaptor.

24. An assembly according to claim 23 wherein, the first adaptor is an angle adaptor having a recess disposed in the first end at an angle relative to its longitudinal axis.

25. An assembly according to claim 24 wherein, the recess in the angle adaptor receives the extension member of the tibial tray allowing the tray to rotate up to 360 degrees.

26. An assembly according to claim 25 wherein rotation of the extension member in the angle adaptor allows the tibial tray to be placed in a variety of selected orientations.

27. An assembly according to claim 26 wherein the stem comprises locating ribs which provide resistance to unwanted rotation of said stem relative to a wall of said medullary cavity.

28. An assembly according to claim 26 wherein the stem comprises first and second spaced apart threads which anchor said stem to said medullary cavity.

29. An angle adaptor for a modular prosthesis assembly insertable in bone, the assembly including:
a tray element having a first surface including an extension member depending therefrom and an opposite second surface;
a position adaptor comprising, a first end and a second end; the first end engagable with the second end of the angle adaptor,
a stem having first and second end, the first end including a formation which allows engagement of said stem with the second end of said position adaptor,
the second end of said stem having a profile suitable for insertion in bone; wherein the angle adaptor has first and second ends, the first end having a profile part which receives and retains the extension member.

28. (canceled)

29. (canceled)

30. An angle adaptor according to claim 29 wherein, the tray is rotatable relative to the angle and position adaptors.

31. An angle adaptor according to claim 30 wherein, the angle adaptor is rotatable relative to the position adaptor.

32. An angle adaptor according to claim 31 wherein the position adaptor is rotatable relative to the stem.

33. An angle adaptor according to claim 32, wherein the second end of the position adaptor engages the first end of the stem via opposite gender interfitting.

34. An angle adaptor according to claim 33, wherein, when the position adaptor is fitted to the stem, a longitudinal axis of the stem aligns with the second longitudinal axis of the second end of the second adaptor.

35. An angle adaptor according to claim 34 wherein the position adaptor engages the stem via recess in the stem.

36. An angle adaptor according to claim 35 wherein the longitudinal axis of the second end of the position adaptor remains off set from the longitudinal axis of the stem.

37. An angle adaptor according to claim 36 wherein, the angle adaptor engages via its second end a recess in the first end of the position adaptor.

38. An angle adaptor according to claim 37 wherein, the angle adaptor is rotatable relative to said recess of the first end of the position adaptor.

39. An angle adaptor according to claim 38 including a recess at its first end which receives and retains therein said extension member of said tray.

40. An angle adaptor according to claim 39 wherein, the recess is disposed at an angle relative to a longitudinal axis of the angle adaptor.

41. An angle adaptor according to claim 40 wherein a longitudinal axis of the recess in the angle adaptor aligns with a longitudinal axis of said extension member of said tray.

42. An angle adaptor according to claim 41 wherein engagement between said extension member of said tray with the angle adaptor and engagement of said angle adaptor with said position adaptor allows a plurality of positional adjustments to the orientation of said tray relative to the longitudinal axis of said stem.

43. An angle adaptor according to claim 42 wherein the tray may be rotationally adjusted relative to the stem via the angle adaptor and relative to the position adaptor via the angle adaptor.

44. An angle adaptor according to claim 43 wherein the tray is a tibial tray for use in an artificial knee.

45. An angle adaptor according to claim 44 wherein, the angle adaptor allows angle and rotational adjustments to the extension member of the tibial tray.

46. An angle adaptor according to claim 45 wherein, the tray provides a tibial component for an artificial knee.

47. An angle adaptor according to claim 46 wherein the stem is insertable in a medullary cavity of a tibia.

48. An angle adaptor according to claim 47 wherein the longitudinal axis of the second end of the position adaptor is offset 1-10 mm from the longitudinal axis of the first end of the position adaptor thereby causing the longitudinal axis of the angle adaptor to be off set from the longitudinal axis of the stem.

49. An angle adaptor according to claim 48 wherein rotation of the extension member in the angle adaptor allows the tibial tray to be placed in a variety of selected orientations.

50. An angle adaptor according to claim 49 wherein, the stem comprises locating ribs which provide resistance to unwanted rotation of said stem relative to a wall of said medullary cavity.

51. An angle adaptor according to claim 50 wherein the stem comprises first and second spaced apart threads which anchor said stem to said medullary cavity.

52. A position adaptor for a modular prosthesis assembly insertable in bone, the assembly including:
a tray element having a first surface including an extension member depending therefrom and an opposite second surface;
an angle adaptor comprising, a first end and a second end; the first end engagable with the extension member,
a stem having first and second end, the first end including a formation which allows engagement of said stem with the second end of said position adaptor,
the second end of said stem having a profile suitable for insertion in bone; wherein the angle adaptor has first and second ends, the first end having a profile part which receives and retains the extension member.

53. A modular prosthesis assembly, the assembly comprising:
a tibial tray element having a first surface and a second, opposite surface, the first surface including an extension member depending therefrom;
an angle adaptor having first and second ends wherein the first end comprises an opening which receives and retains therein the extension member;
the position adaptor comprising, a first end and a second end; a first longitudinal axis extending through the first end which is substantially parallel to but offset from a second longitudinal axis extending through the second end, the first end having a recess matable with the second end of the angle adaptor; the second end of the position adaptor adapted for mating with an elongate stem mountable within bone, the elongate stem having a first end that is selectively matable with the second end of the position adaptor; wherein the angle adaptor allows angle adjustments and the position adaptor allows rotational positional adjustments effective to secure the position adapter in a desired orientation when mated with the angle adaptor and stem.

54. A positional adaptor for use with a modular tibial prosthesis assembly, the assembly comprising:
a tray element having a first surface that is mountable upon bone and a second, opposed surface, the first surface including an extension member depending therefrom;
the position adaptor matable with said extension member and comprising , a first end and a second end; a first longitudinal axis extending through the first end which is substantially parallel to but offset from a second longitudinal axis extending through the second end, the first end having a recess matable with the extension member the second end adapted for mating with an elongate stem mountable within bone, the elongate stem having a first end that is selectively matable with the second end of the position adaptor; wherein the position adaptor allows rotational positional adjustments effective to secure the position adapter in a desired orientation when mated with the stem.

55. A positional adaptor according to claim 54 wherein the second end of the positional adaptor mates with the stem in a male female relationship.

56. A positional adaptor according to claim 55 wherein the first end of the angle adaptor has a female recess and the second end is a male profile part.

57. A positional adaptor according to claim 56 wherein the tray is a tibial plate and the extension member is a tibial stem.

58. A positional adaptor according to claim 57 wherein the first and second longitudinal axes of the position adaptor are offset from each other by approximately 1 to 10 mm.

59. A positional adaptor according to claim 58 wherein the first and second ends of the position adaptor are generally cylindrical, the first end having a tapered recess to accommodate the angle adaptor and the second end having a male profile part.

60. A position adaptor for connection between two components of a tibial joint prosthesis, the position adaptor comprising:
a body having first and second ends wherein a first longitudinal axis extending through the first end is substantially parallel to but offset from a second longitudinal axis extending through the second end;
a first connection surface integral with the first end that is adapted to mate with a first tibial joint prosthesis component; and a second connection surface integral with the second end that is adapted to mate with a second joint prosthesis component;
a first component comprising an extension member depending from a tibial plate and the second component comprising a stem matable with tibial bone;
the adaptor element being effective to join the first and second joint prosthesis components together such that a longitudinal axis extending through the second joint prosthesis component is offset from a longitudinal axis extending through the first joint prosthesis component, wherein the first adaptor is rotatable relative to the first and second components to allow positional adjustment of one axis of said adaptor between 1-10 mm.

61. A modular prosthesis assembly comprising; an anchorage component insertable in bone and a coupling component which cooperates with said anchorage component to assume a first predetermined orientation relative to said anchorage component; the assembly further comprising an adaptor insertable between said anchorage component and said coupling component to allow a secondary adjustment of said coupling component relative to said first predetermined orientation of said coupling component.

62. A modular prosthesis assembly comprising;
an anchorage component insertable in a bone cavity,
a position adaptor for coupling to the anchorage component and having a first end which is off set from a longitudinal axis of the anchorage component and a second end in alignment with said axis and which engages the component so as to allow up to 360 degrees of relative rotation therebetween;
an angle adaptor having a longitudinal axis and first and second ends, the second end engaging the first end of the position adaptor and allowing up to 360 degrees of rotation relative to the position adaptor prior to fixed engagement between the angle adaptor and the position adaptor, the first end of the angle adaptor including a recess disposed at an angle to its longitudinal axis,
a tray including an extension stem which is received and retained by said angled recess; wherein the tray is rotatable through up to 360 degrees relative to the angle adaptor; wherein engagement between the position adaptor and stem and the angle adaptor and position adaptor and between said extension stem and angle adaptor allow the tray to undergo fine adjustments including rotation in an X plane and Y plane and rotation about a Z axis.

63. A modular prosthesis assembly according to claim 62 wherein the offset in said position adaptor and the angled recess in said angle adaptor, when the position adaptor is coupled to the angle adaptor, cooperate to allow said rotational movements.

64. A modular prosthesis assembly according to claim 63 wherein said tray is capable of rotation about and at a selected distance from the longitudinal axis of said anchor member.

65. A prosthesis assembly according to claim 1 wherein the angle adaptor and position adaptor are arranged to provide fine adjustments in orthogonal X and Y planes and a Z plane to joints selected from dental fixations, tibial and femoral implants (distal or proximal), ankles, shoulders, fingers and thumbs.

66. A method of insertion of a modular tibial prosthesis assembly, comprising the steps of
a) taking a tray element having a first surface that is mountable upon bone and a second, opposed surface, the first surface including an extension member depending therefrom;
b) taking an angle adapter element having first and second ends wherein the first end comprises an opening which receives and retains therein the extension member;
c) taking a position adaptor comprising, a first end and a second end; a first longitudinal axis extending through the first end which is substantially parallel to but offset from a second longitudinal axis extending through the second end,
d) taking a stem capable of insertion in bone;
d) inserting the stem in a medullary cavity of a tibia;
e) inserting the positional adaptor via its second end into the stem, the elongate stem having a first end that is selectively matable with the second end of the position adaptor;
f) placing the angle adaptor via its second end in a recess is the first end of the position adaptor
g) inserting the extension member of the tibial plate into a recess in said angle adaptor via its first end
h) adjusting the tibial plate by selective adjustment of the angle adaptor and the position adaptor to orientate the tibial plate;
i) adjusting the position adaptor within an arc of 0-360 degrees.
wherein the angle adaptor allows angle adjustments and the position adaptor allows rotational positional adjustments effective to secure the tibial plate in a desired orientation when mated with the assembly.

67. A method according to claim 66 comprising the further additional steps of:
a) checking the orientation of the tibial plate relative to a predetermined anatomical reference once the angle adaptor and position adaptor have been assembled;
b) in the event that the first orientation of the tibial plate is incorrect relative to said anatomical reference, resetting said plate and either the angle and/or position adaptor so the tibial plate assumes a secondary disposition which is a preferred orientation relative to a predetermined anatomical reference.

68. An angle adaptor according to claim 29 wherein the second end of said position adaptor is offset from the first end.

69. An angle adaptor according to claim 68 wherein the position adaptor includes a first longitudinal axis extending through its first end and a second longitudinal axis offset from the first longitudinal axis and extending through the second end.
